(19) United States
(12) Patent Application Publication
(10) Pub. No.: US 2005/0244354 A1
(43) Pub. Date: Nov. 3, 2005

(54) Oxygenated personal care products

(76) Inventor: Sam Speron, Park Ridge, IL (US)

Correspondence Address: ADAM K. SACHAROFF;MUCH SHELIST FREED DENENBERG AMENT&RUBENSTEIN,PC, 191 N. WACKER DRIVE, SUITE 1800, CHICAGO, IL, 60606-1615, US

(21) Appl. No.: 10/835,150

(22) Filed: Apr. 30, 2004

(51) Int. Cl. A61K 7/06

(52) U.S. Cl. 424/70.1

(57) ABSTRACT

The present invention relates to a personal care composition containing water that has been treated with a combination of ozone and hydrogen peroxide. The personal care composition is further treated with liquid oxygen that is added to the composition prior to packaging. The personal care composition may be in the form of a soap, lotion, cream, balm, stick, foundation, spray, mousse, gel, sanitizer, wash, or towellete, and directed for various uses on the human body. The ozone and hydrogen peroxide is added to the water prior to manufacturing the composition.

### BACKGROUND OF THE INVENTION

The present invention relates to products directed to personal care products and more particularly to a composition for oxygenated rich personal care products.

The prior art is crowded with personal care products, many of which are intended for clinical and domestic use. In testing, it has been found that many personal care products on the market today do not provide an efficacy to provide an increased amount of oxygen rich elements back into the skin and body. Oxygen is the most vital nutrient required by the human body for cell reproduction and growth. Studies have shown that the most significant factor in health and longevity is how well you breathe. Unfortunately, today's breathable air contains less that 22% oxygen, while at one time our breathable air contained more than 50% oxygen.

The answer is that we have adapted but at a cost. That cost is evident in the number of cases of cancer, AIDS, heart attacks and other illnesses that cripple and kill our bodies. The facts are that without an adequate amount of oxygen in our bodies our cells become less efficient in reproduction and growth and start to turn into sick cells. The more sick cells a person accumulates, the better the chances of that person becoming terminally ill. If you are living in either a big city or at a high altitude, then you are experiencing an even worse air quality with a respective lack of oxygen.

Further studies have shown that bacteria, viruses, and diseases find it very difficult to live and survive in an oxygen rich environment. Furthermore oxygen has been shown to have rejuvenative effects, especially on the skin. The facts are that if our skin can be exposed to more oxygen, our skin cells will reproduce and grow healthier, which will enable our skin to become stronger and stay stronger.

Possible benefits from oxygen therapy may include, but are not limited to, the following: (1) overall health benefits in reducing stress, preventing symptoms of old age, maintaining the body's efficiency, and stimulating the immune system for people prone to disease; (2) skin benefits in alleviating pain and reducing recovering time for burns, wounds, pressure areas, and open wounds, regenerating skin cells, cleaning skin, clearing acne and scars caused from acne, reducing and clearing wrinkles, revitalizing hair cells, and clearing up cellulites; (3) benefits to the body's conservation and regulation in alleviating problems with hormone regulation (menstrual problems, impotence, menopause) and reducing exhaustion, convalescence, chronic illnesses, and the visual effects of old age; (4) benefits to rheumatic illnesses may include reducing pain and inflammation caused by rheumatism, polyarthritis, and arthritis; (5) increasing blood supply or oxygen supply to the brain to help reduce headaches/migraines, strokes, and problems with sleeping, concentration, memory, tiredness, and dizziness; (6) increasing blood supply to the heart to help reduce angina, pectoris, and heart attacks; (7) reduce circulation problems caused by low or high blood pressure, and reduce micro-circulatory conditions (e.g. retinal eye problems); (8) increasing blood supply to the extremities to reduce lack of feeling, symptoms with feelings of heavy legs, cramps, and varicose veins; (9) benefits to therapy for cancer illnesses in the reduction in the side effects of chemotherapy and radiation therapy; (10) helps rejuvenate body tissues and flushes toxins from the tissue's cells, help repair damaged tissue, slows the aging process, combats signs of stress, and helps eliminate skin conditions (acne, eczema, psoriasis); (11) reduces scarring by accelerating the healing process, boosting the immune system to combat possible infection, improving cellular regeneration, and allowing faster & cleaner reconstruction of lost and damaged tissue; and (12) helps alleviate pain and symptoms associated with organic and metabolic problems such as high fat values, high cholesterol and uric acid values, illnesses resulting from diabetes, and hepatitis or liver cirrhosis.

A need therefore exists to provide oxygenated rich personal care products that when used regularly a person may have one or more of the benefits listed above.

### SUMMARY OF THE INVENTION

The present invention is directed to personal care such as skin and body products that incorporate oxygenated purified water that increases the oxygen content on the body and helps rejuvenate the body. The personal care “products” may include soaps, creams, wipes or towelettes, washes, shampoos, conditioners, sprays, lotions, balms, enamels, tonics, and/or mousses. These “products” may be specifically created for the face, hands, body, hair, eyes, or lips. In addition, the products may be further enriched with other known ingredients such as surfactants, detergents, or emollients, such that the products could be otherwise directed for specific intended uses such as sun screens, sunless tans, moisturizers, acne prevention, sanitizers, or hydrocortisone products. In addition drinking water or cough drops may be enriched with oxygen to increase internal body compositions. The products identified herein are based on known soaps, creams, lotions, etc., and have a substrate or carrier. The products are furthermore made according to the prior art except for the added steps or ingredients to provide an oxygen rich product.

### DETAILED DESCRIPTION OF THE INVENTION

The present invention includes a personal care product that has a carrier substrate made in according to its specific prior art. For example, if the personal care product is soap, the personal care product is made in accordance to well known soap processes to create a carrier substrate. In the process of making the personal care product, oxygen rich ingredients are added to the personal care product's composition or carrier to increase the free oxygen atoms in the personal care product. In addition, any water that is used in making the personal care product is preferably purified water, which may be purified by known methods in the art such as de-ionization, reverse osmosis, boiling, etc. However, the water may be non-purified for a less desirable or effective product. Prior to using the water in the manufacturing process of the personal care product, the water is ozonated in accordance to different methods known in the art. In one preferred method, the water is enriched with ozone and hydrogen peroxide. It has been found that since hydrogen peroxide and ozone are both relatively unstable aggregations of atoms of oxygen, these two ingredients will interact to dissociate and increase the number of free oxygen atoms in the water. The process of manufacturing the personal care product is then commenced under normal or typical prior art conditions. Upon completing the process of manufacturing the personal care product a stream of liquid oxygen is added into the personal care product. This significantly increases the Oxygen content of the final personal care product.

In but one process the water is enriched with ozone at a rate between about 1 liter to about 10 liters per 100 gallons of water, more preferably at a rate between about 3 liters to about 7 liters per 100 gallons of water, and most preferably at a rate about 6 liters per 100 gallons of water. Hydrogen peroxide is also added preferably at a rate between about 0.5 liters to about 2 liters per 100 gallons of water and most preferably at a rate of about 1 liter per 100 gallons of water. The personal care product is lastly infused with a stream of liquid oxygen preferably at a rate of between about 1 liter to about 8 liters per 100 gallons of finished personal care product, more preferably at a rate between about 2 liters to about 6 liters per 100 gallons of finished personal care product, and most preferably at a rate about 4 liters per 100 gallons of finished personal care product. The rates of ozone, hydrogen peroxide and liquid oxygen may be increased or decreased depending upon the amount of oxygenation desired in the finished personal care product. These rates are applicable throughout the different processes for the different personal care products.

The compositions of the personal care products may vary depending upon intended use or effect the personal care product. In one instance, the composition further comprises a nutrient, such as vitamins, essential amino acids and essential fatty acids. Examples of such vitamins include, but are not limited to vitamin A, vitamin B, vitamin C, vitamin D, and vitamin E. Examples of such essential amino acids include, but are not limited to, arginie, histidine, isoleucine, leucine, lysine, methionine, phenylalanine, threonine, tryptophan, and valine. Examples of such essential fatty acids, include, but are not limited to, linoleate and linolenate.

The compositions of the personal care products may further includes a skin-conditioning agent selected from the group consisting of emollients and humectants. Emollients help maintain the soft, smooth, and pliable appearance of the skin, while humectant is intended to increase the water content of the top layers of the skin. Examples of emollients include plant, nut, and vegetable oils. Examples of humectants include glycerin or trehalose or a salt or ester thereof.

The compositions of the personal care products may further include other cosmetically active agents that has a cosmetic or therapeutic effect on the skin, e.g. agents to treat wrinkles, acne, or pigmentation to lighten the skin. In one embodiment, the agent is selected, but not limited to, from the group consisting of hydroxyl acids, benzoyl peroxide, sulfur resorcinol, ascorbic acid, D-panthenol, hydroquinone, sun screen agents, anti-inflammatory agents, pigmentation or skin lightening agents, antimicrobial and antifungal agents, estrogens, 2-dimethylaminoethanol, lipoic acid, amino acids (such as praline and tyrosine), lactobionic acid, acetyl-coenzyme A, niacin, riboflavin, thiamin, ribose, electron transporters, botanical extracts such as aloe vera, herbal, and soy and derivatives and mixtures thereof.

The personal care product composition can be used by topically administering it, by direct laying, spreading, or spraying on the skin or hair of a human. The topical compositions useful in the present invention may be used for a variety of cosmetic uses, including, but not limited to, treating, cleansing, beautifying, or covering the skin or hair. The compositions, thus, may be made into a wide variety of product types. These include, but are not limited to, soaps, lotions, creams, gels, sticks, sprays, ointments, pastes, mousses, shampoos, cosmetics, dermal patches, wipes, washes, or towelettes. Products include, but are not limited to, hand soaps, bar soaps, body washes, herbal body washes, anti-aging body lotions, aloe vera lotions and creams, hand repair creams, hydrocortisone creams, moisturizing body lotions, firming body lotions, hand sanitizers, antibacterial towelletes, sun treatment compositions, such as but not limited to, sunless tanning lotions, sun damage repair cream, and sun block, hair treatment compositions, such as but not limited to, shampoo, conditioners, shampoo and conditioner mixtures, leave in conditioners, hair gels, hair tonic, hair creams, and hair stylist mousses, acne treatment compositions, such as but not limited to, acne washes with salicyclic acid, acne cleaners with benzoyl peroxide, and acne scar cream, face treatment compositions, such as but not limited to, facial cleaners, facial toners, pigmentation control compositions, face wipes, eye make up removers, lip balms, lip repair creams, anti-agining eye creams, rosacea and redness creams, facial moisturizers and ficial exfoliates, and other types of known moisturizers, lotions, creams, scrubs, and body mists. Other personal care products which may be derivatives of the above may include skin toners, face and body creams and gels, face and body firming lotions, hydrating cleaners, antiperspirants, nail enamels, lipsticks, foundations, or mascaras. The products may includes several types of carrier systems that may be made in accordance with the prior art. If the personal care product is formulated as an aerosol and applied to the skin as a spray-on, a propellant is added to a solution composition. Examples of propellants include, but are not limited to, chlorinated, fluorinated, and chloro-fluorinated lower molecular weight hydrocarbons.

In a first embodiment the personal care product is soap. The soap may be hard or soft, solid or liquid, as is known in the prior art. The soap may be composed of ingredients such that the soap is preferably used on the hands, body, or face. The soap composition includes a soap carrier and a combination of ozone, hydrogen peroxide and liquid oxygen. As mentioned above, the ozone and hydrogen peroxide are added to water at the beginning phase of the manufacturing process. The liquid oxygen is then added near the end phase of the manufacturing process, preferably prior to final packaging (if the personal care product is soft or liquid) or prior to hardening (if the personal care product is hard or solid). A benefit to the soap in accordance with the present invention is that the hands, body, or face is also being treated with oxygenation that may help rejuvenate, restore, or treat the skin's cells. It being well known that if the soap is intended to be more liquid, as in a wash or lotion, additional oxygenated water would be added to the product. In addition the soap could be saturated or placed onto wipes or towelettes. In addition, if the soap is intended to be a lotion or cream additional agents may be added to thicken but not harden the soap. Other agents such as aloe Vera, hydrocortisone, firming agents and other moisturizing agents may also be added to the soap for create various products.

In a second embodiment a personal care product is a hair treatment product, such as but not limited to shampoo, conditioner, combinations thereof, or a hair spray, gel, mousse, creams, tonics, or other type of stylist. The hair treatment carrier may include a sudsing agent in an amount of between 5% and 50% by weight of the composition. While various sudsing agents are contemplated and well known in the art, the present embodiment may include ammonium lauryl sulfate, sodium lauryl sulfate, or sodium laureth sulfate. The sudsing agent assists with cleaning by cutting grease, grime and dirt from the hair, scalp, skin and other surfaces. The hair treatment composition would include the hair treatment carrier and a combination of ozone, hydrogen peroxide and liquid oxygen. As mentioned above, the ozone and hydrogen peroxide are added to water at the beginning phase of the manufacturing process. The liquid oxygen is then added near the end phase of the manufacturing process, preferably prior final packaging of the hair treatment. A benefit of the hair treatment product in accordance with the present invention is that the hair, scalp, and skin are being treated with oxygenation that may help rejuvenate, restore, or treat the user's cells.

In a third embodiment a personal care product is a liquid sanitizer, such as a hand, facial or body wash. The liquid sanitizer carrier will typically include a large amount of alcohol in some instances up to 70% by weight of the composition or higher. The alcohol may be isopropyl alcohol or SDA 40. In addition to other surfactants, detergents, emollients, and/or alcohols included in the sanitizer, the product in accordance to the second embodiment will include a predetermined amount of water. The water is as mentioned treated with ozone and hydrogen peroxide. Furthermore liquid oxygen is added to the sanitizer prior to final packaging. A benefit of the liquid sanitizer made in accordance with the present invention is the liquid sanitizer helps rejuvenate, restore, or treat the skin cells with oxygen.

In a fourth embodiment a personal care product is a sun treatment product, which as known in the art may include a carrier that is either a lotion or cream. The sun treatment product may be directed to a sunless tanning composition, a sun-screening composition, or a sun damage cream. In the sun-screening composition a carrier will typically include octy methoxycinnamate in an amount of between 2% to about 13% and preferably 5% to 9% by weight of the composition; benzophenone 3 in an amount of between 1% and 11% and preferably between 4% and 8% by weight of the composition; and include homomethyl salicylate in an amount of between 5% and 15% and preferably between 8% and 12%. In a sun damage cream, a carrier will include various vitamins such as vitamin A and/or C, or other sun damage repair agents known in the art. In a sunless tanning composition, a carrier may include active ingredients such as but not limited to dihydroxyacetone. Most of the remaining portion of the carriers is made up of various oils, vitamins and other known ingredients. The portion of the compositions that is water includes ozone and hydrogen peroxide. Furthermore liquid oxygen is added to the sun-screen composition prior to final packaging. A benefit of the sun-screen made in accordance with the present invention is the sun-screen will help replace, rejuvenate and/or restore the skin cells with oxygen. In addition, oxygen is a known treatment for burns, therefore the products may help lessen the effects of burning.

The fifth embodiment of the present invention is directed to moisturizing personal care products such as but not limited to facial, hand, lips, or body moisturizing lotions, creams, or balms. The moisturizing products include a moisturizing carrier that may include different blends of antioxidants, vitamins, oils, Aloe Vera, proteins, propylene glycol, and/or acids. As mentioned above, the water used in the products include ozone and hydrogen peroxide. Liquid oxygen is added to the moisturizing product prior to final packaging. A benefit of the moisturizing products made in accordance with the present invention is the moisturizing products help replace, rejuvenate, restore and increase the oxygen content on and within the cells.

The sixth embodiment of the present invention the personal care is directed to acne treatment compositions such as but not limited to acne washes and cleansers and acne scar treatment compositions. Some of these compositions are used to remove accumulated oil, make-up, sweat, and dirt on the surface of the skin. The acne cleansers and washers include different carriers that may include active ingredients to help break down keratin in blackheads, such ingredients may include, but are not limited to, salicylic acid and benzoyl peroxide (which also has antibacterial effects). The acne scar compositions may have carriers that include active ingredients such as but not limited to different vitamins, minerals, acids and alcohols. As mentioned above, the water used in the personal care products directed to acne cleaners and washers include ozone and hydrogen peroxide. Liquid oxygen is added prior to final packaging.

The seventh embodiment of the personal care products is directed to face treatment composition such as but not limited to facial cleaners, toners, pigment controls, wipes, exfoliates, rosacea and redness repairs, and foundations, eye make up removes, lip balms, lip repair creams, and anti-aging eye creams. The face treatment compositions will include various carriers with active ingredients directed to the specific personal care product. As mentioned above, the water used in the personal care products directed to acne cleaners and washers include ozone and hydrogen peroxide. Liquid oxygen is added prior to final packaging.

In an eight embodiment of the present invention, the personal care products may not include water are enough water such that the products are not sufficiently oxygenated. In these instances, an increased amount of liquid oxygen is added to the personal care products and sufficiently mixed to allow the liquid oxygen to infuse with the product. The amount of liquid oxygen may vary but in one instance the liquid oxygen is preferably added at a rate of between about 2 liters to about 6 liters per 100 gallons of finished personal care product and most preferably at a rate between about 4 liters per 100 gallons of finished personal care product.

The mild and gentle blends of surfactants, detergents, emollients, and alcohols used by the carriers may include, but are not limited to, some of these ingredients: Acelated Lanolin Alcohol, Alcohols, Alfalfa, Allantoin, Almond Oil, Aloe Oil, Aloe Vera, Aloe Vera Gel, Benzophenone 3, Benzophenone 4, Benzyl Nicotinate, Burdock Root, Calcium Carbonate, Calendula Flower, Canola Oil, Carbomer, Carbomer 940, Carbowax 400, Cetearyl Alcohol, Cetyl Alcohol, Chamomile, Chlorophyll, Citric Acid, Cocamide MEA, Cocamidopropyl Betaine, Coco Butter, Cocoamidopropyl Betaine, Cocoglucoside, Coconut Oil, Colorants, Cyclomethicone, Cyclopentasiloxane, Dimethicone, Dimethicone Copolyol, Disodium EDTA, DL Panthenol, DMDM hydantoin, DMDM Hydantoin, Green Tea, Flax Seed, Glycerin, Glycerol Sterate, Glyceryl Oleate, Glyceryl Stearate, Glycol Stearate, Grape Fruit Seed, Green Tea, Homomethyl Salicylate, Hydrocortisone, Isopropyl Palmitate, Jojoba Oil, Laureth 7, Lauryl Glucoside, Lidocane, Magnesium Sulfate, Marygold, Menthol Crystals, Methylparaben, Mineral Oil, Mint, Neem, Nettles, Oat Protein, Octyl Methoxycinnamate, Octyl Stearate, Orange Peel, Panthenol, PEG 30 Stearate, Peppermint Oil, Phenoxyethanol, Polyquaternium 10, Polyquaternium 11, Polyquaternium 7, Polysorbate 20, Polysorbate 60, Polysorbate 80, Potassium Sorbate, PPG-2 Myristyl Ether Propionate, Propylene Glycol, Propylparaben, Rosemary, Sage, SDA 40, Shea Butter, Silica, Sodium C-14-16 Olefin Sulfonate, Sodium Chloride, Sodium Citrate, Sodium Lactate, Sodium Laureth Sulfate, Sodium Lauryl Ether Sulfate, Sodium Lauryl Sulfate, Sodium PCA, Sodium Silicate, Sorbitol, Soy Protein, Stearic Acid, Stearyl Alcohol, Sulfated Castor Oil, Tetrasodium EDTA, Triethanolamine, Vitamin A, Vitamin C, Vitamin D, Vitamin E, Watercress, Wheat Protein, Witch Hazel Extract, Xanthan Gum, and Zinc Oxide.

In yet another embodiment present oxygenated water is bottled and sold as a drinkable water. In this embodiment the drinking water is preferably purified by reverse osmosis and then ozone and hydrogen peroxide is added in the amounts previously specified. The drinking water is then preferably treated to create a carrier. The treatment is preferably done with minerals, favors, and vitamins, such as but not limited to, the following: sulfated Castor Oil, Aloe Oil, Magnesium Sulfate, Calcium Carbonate, Sodium Cholide, Sodium Silicate, Methylparaben, Propylparaben Vitamin A&D, Vitamin C, Vitamin E, Benzyl Nicotinate, Peppermint Oil. The drinking water is finally processed with liquid oxygen prior to final packaging.

From the foregoing and as mentioned above, it will be observed that numerous variations and modifications may be effected without departing from the spirit and scope of the novel concept of the invention. It is to be understood that no limitation with respect to the specific embodiments illustrated herein is intended or should be inferred. It is, of course, intended to cover by the appended claims all such modifications as fall within the scope of the claims.

1. A personal care composition comprising:
a carrier substrate and
liquid oxygen.

2. The personal care composition according to claim 1, wherein said personal care composition comprises a soap, lotion, cream, balm, stick, foundation, spray, mousse, gel, sanitizer, wash, wipe, shampoo, conditioner, enamel, tonic or towellete.

3. The personal care composition of claim 1 wherein said carrier substrate includes water and further comprising ozone and hydrogen peroxide added to said water.

4. The personal care composition according to claim 3, wherein said ozone is added to said water at a rate of between about 1 liter and about 10 liters per about 100 gallons of water.

5. The personal care composition according to claim 3, wherein said ozone is added to said water at a rate of between about 3 liters to about 7 liters per about 100 gallons of water.

6. The personal care composition according to claim 3, wherein said ozone is added to said water at a rate of about 6 liters per about 100 gallons of water.

7. The personal care composition according to claim 3, wherein said hydrogen peroxide is added to said water at a rate of between about 0.1 liters to about 3 liters per about 100 gallons of water.

8. The personal care composition according to claim 3, wherein said hydrogen peroxide is added to said water at a rate of about 1 liter per about 100 gallons of water.

9. The personal care composition according to claim 1, wherein said liquid oxygen is infused into said carrier substrate at a rate of between about 1 liter to about 8 liters per about 100 gallons of said personal care composition.

10. The personal care composition according to claim 1, wherein said liquid oxygen is infused into said carrier substrate at a rate of between about 3 liters to about 5 liters per about 100 gallons of said personal care composition.

11. The personal care composition according to claim 1, wherein said liquid oxygen is infused into said carrier substrate at a rate of about 4 liters per about 100 gallons of said personal care composition.

12. A soap composition comprising:
a soap carrier; and
liquid oxygen.

13. The soap composition of claim 12, wherein said soap carrier includes water and further comprising ozone and hydrogen peroxide added to water.

14. The soap composition of claim 13 wherein said ozone and hydrogen peroxide are added to water to define ozonated water.

15. The soap composition of claim 13, wherein said ozone is added to said water at a rate of between about 4 liters and about 8 liters per about 100 gallons of water.

16. The soap composition of claim 15, wherein said hydrogen peroxide is added to said water at a rate of between about 0.5 liters to about 2 liters per about 100 gallons of water.

17. The soap composition of claim 12, wherein said liquid oxygen is infused into said carrier substrate at a rate of between about 2 liters to about 6 liters per about 100 gallons of said soap composition.

18. The soap composition of claim 13, wherein said water is oxygenated purified water.

19. The soap composition of claim 18, wherein the oxygenated purified water is infused with liquid oxygen.

20. The soap composition of claim 19 wherein the oxygenated purified water further contains ozone and hydrogen peroxide.

21. A hair treatment composition comprising:
a hair treatment carrier; and
liquid oxygen infused into said hair treatment carrier.

22. The hair treatment composition of claim 21, wherein the hair treatment carrier includes water and further comprising ozone and hydrogen peroxide added to said water.

23. The hair treatment composition of claim 22 wherein said hair treatment composition is a shampoo, conditioner, shampoo and conditioner mixture, hair spray, gel, mousse, cream, or tonic.

24. The hair treatment composition of claim 22, wherein said ozone is added to said water at a rate of between about 4 liters and about 8 liters per about 100 gallons of water.

25. The hair treatment composition of claim 22, wherein said hydrogen peroxide is added to said water at a rate of between about 0.5 liters to about 2 liters per about 100 gallons of water.

26. The hair treatment composition of claim 21, wherein said liquid oxygen is infused into said hair treatment carrier at a rate of between about 2 liters to about 6 liters per about 100 gallons of said hair treatment composition.

27. A sun treatment composition comprising:
a sun treatment carrier; and
liquid oxygen.

28. The sun treatment composition of claim 27, wherein the sun treatment carrier includes water, and further comprising ozone and hydrogen peroxide added to said water.

29. The sun treatment composition of claim 27 wherein the sun treatment carrier is selected from the group consisting of a sunless tanning carrier, a sun-screening carrier, or a sun damage repair carrier.

30. The sun treatment composition of claim 28, wherein said ozone is added to said water at a rate of between about 4 liters and about 8 liters per about 100 gallons of water.

31. The sun treatment composition of claim 28, wherein said hydrogen peroxide is added to said water at a rate of between about 0.5 liters to about 2 liters per about 100 gallons of water.

32. The sun treatment composition of claim 28, wherein said liquid oxygen is infused into said sun treatment carrier at a rate of between about 2 liters to about 6 liters per about 100 gallons of said sun treatment composition.

33. The sun treatment composition of claim 28, wherein the sun treatment carrier includes octy methoxycinnamate, benzophenone 3, or homomethyl salicylate.

34. The sun treatment composition of claim 28, wherein the sun treatment carrier includes dihydroxyacetone.

35. The sun treatment composition of claim 28, wherein the sun treatment carrier includes vitamin A, or vitamin C.

36. An acne treatment composition comprising:
an acne treatment carrier; and
liquid oxygen.

37. The acne treatment composition of claim 36, wherein the acne treatment carrier includes water and further comprising ozone and hydrogen peroxide added to said water.

38. The acne treatment composition of claim 37 wherein the water is ozonated.

39. The acne treatment composition of claim 37, wherein said ozone is added to said water at a rate of between about 4 liters and about 8 liters per about 100 gallons of water.

40. The acne treatment composition of claim 37, wherein said hydrogen peroxide is added to said water at a rate of between about 0.5 liters to about 2 liters per about 100 gallons of water.

41. The acne treatment composition of claim 37, wherein said liquid oxygen is infused into said acne treatment carrier at a rate of between about 2 liters to about 6 liters per about 100 gallons of said acne treatment composition.

42. The acne treatment composition of claim 38, wherein the acne treatment carrier includes salicylic acid, or benzoyl peroxide.

43. The acne treatment composition of claim 42, wherein the acne treatment carrier includes an acne scarring repair agent.

44. A personal care composition in the form of a soap, lotion, cream, balm, stick, foundation, spray, mousse, gel, sanitizer, wash, or towellete, the composition is further infused with liquid oxygen.

45. The personal care composition of claim 44 further comprising water that has a combination of ozone and hydrogen peroxide.

46. A face treatment composition comprising:
a face treatment carrier; and
liquid oxygen.

47. The face treatment composition of claim 46, wherein the face treatment carrier includes water and further comprising ozone and hydrogen peroxide added to said water.

48. The face treatment composition of claim 47, wherein the water is defined as an ozonated water.

49. The face treatment composition of claim 46, wherein the face treatment carrier includes a soap, sanitizer, moisturizer, toner, pigment control agent, an exfoliate, rosacea and redness repairer agent, lip repair cream, or anti-aging cream.

50. A drinking water composition comprising:
a water carrier;
a combination of ozone and hydrogen peroxide; and
liquid oxygen infused into said water carrier.

51. The drinking water composition of claim 50 wherein the water carrier is purified by reverse osmosis.

52. A method of preparing a personal care composition comprising:
providing a carrier substrate and
infusing liquid oxygen into said carrier substrate.

53. The method of claims 52, wherein said personal care composition comprises a soap, lotion, cream, balm, stick, foundation, spray, mousse, gel, sanitizer, wash, wipe, shampoo, conditioner, enamel, tonic or towellete.

54. The method of claim 52 wherein said step of providing said carrier substrate includes adding ozone and hydrogen peroxide to said carrier substrate.

55. A method of preparing a sun treatment composition comprising:
providing a sun treatment carrier; and
infusing liquid oxygen into said sun treatment carrier.

56. The method of claim 55 further comprising a step of, when the sun treatment carrier includes water, adding ozone and hydrogen peroxide to said water.

57. The method of claim 55, wherein the sun treatment carrier is selected from the group consisting of a sunless tanning carrier, a sun-screening carrier, or a sun damage repair carrier.

58. A method of preparing an acne treatment composition comprising:
providing an acne treatment carrier; and
infusing liquid oxygen into said acne treatment carrier.

59. The method of claim 58, further comprising a step of, when the acne treatment carrier includes water, adding ozone and hydrogen peroxide to said water.

60. A method of preparing a face treatment composition comprising:
providing a face treatment carrier; and
infusing liquid oxygen into said face treatment carrier.

61. The method of claim 60, further comprising a step of, when the face treatment carrier includes water, adding ozone and hydrogen peroxide added to said water.

62. The method of claim 60, wherein the face treatment carrier includes a soap, sanitizer, moisturizer, toner, pigment control agent, an exfoliate, rosacea and redness repairer agent, lip repair cream, or anti-aging cream.

63. A method for creating a drinking water composition comprising:
providing a water carrier;
combining ozone and hydrogen peroxide in said water carrier; and
infusing liquid oxygen into said water carrier.

64. The method of claim 63 further comprising the step of purifying the water carrier by reverse osmosis.
